(19) United States
(12) Patent Application Publication
(10) Pub. No.: US 2006/0178660 A1
(43) Pub. Date: Aug. 10, 2006

(54) System for the stimulation of acupuncture points

(75) Inventors: Rolf Neher, Waldsee (DE); Heinz Schade, Reutlingen (DE); Boris Breivogel, Bad Durkheim (DE)

Correspondence Address: Tue Nguyen, 7901 Stoneridge Dr. #415, Pleasanton, CA, 94588, US

(73) Assignee: NS Welltec GmbH, Waldsee (DE)

(21) Appl. No.: 11/351,110

(22) Filed: Feb. 8, 2006

(30) Foreign Application Priority Data
Feb. 8, 2005 (DE) 10 2005 007 906.3
Mar. 24, 2005 (DE) 10 2005 014 586.8

(51) Int. Cl. A61B 18/18

(52) U.S. Cl. 606/9

(57) ABSTRACT

An invention is disclosed for a stimulation therapy utilizing stimulation of acupuncture points by employing a combination of an electrical current and a laser beam in which pulsed stimulation is generally preferred. The invention also comprises an illumination of radiated light for inhibiting the stimulation of nearby acupuncture points, thus enable selective stimulation. The invention can further comprise a stimulation point indicator, showing a sequence of selected body acupuncture points according to the intended treatment effect, which are stimulationable, distinguishable from other points.

This application claims priority from German application No. 10 2005 014 586.8 filed Mar. 24, 2005, entitled “System zur Stimulation von Akupunkturpunkten”, which claimed priority from German application No. 10 2005 007 908.3 filed Feb. 08, 2005, entitled “System zur Stimulation von Akupunkturpunkten” which are incorporated herein by reference.

This application is related to a co-pending application of the same inventors, entitled “System for the stimulation of acupuncture points”.

### FIELD OF THE INVENTION

This invention is related to acupuncture therapy, and particularly related to the stimulation of acupuncture points, with computer-assisted display control.

### BACKGROUND OF THE INVENTION

The effectiveness of acupuncture for various illnesses has been recognized for a long time, and has been proven recently in several studies. Traditionally, specific points in the body, called acupuncture points, were reached by piercing with needles for stimulation. The stimulation can also occur from thermal energy by burning Moxa during treatment, or by manual pressure from fingertips or knuckles (acupressure).

Electrical stimulation of acupuncture points is also well-known, either with alternating or direct, constant or pulsed, current or voltage applied to the piercing needles or directly to the body. In recent years, visible light and laser light are also utilized as a further enhancement of the stimulation of acupuncture points.

Problems with Existing Technologies

The stimulation of acupuncture points with piercing needles can bring risk of infection and is usually not acceptable, especially for children. The same applies to the stimulation of piercing needles with electrical current. Laser light stimulation with different frequencies provides a small effect on acupuncture therapy, but this effect is realized only with much experiences or practice. For electrical stimulation, there is an indeterminable variation of pulse waveforms and frequencies, and thus so far no reliable studies have admitted to the predictability of the effect of the electrical stimulation of acupuncture points. There are indirect proofs from the electrical physiology realizations over the fact that nerve actions can be blocked by high frequencies and activated by low frequencies (1-100 Hertz), however acupuncture point was not proven to be an information line over nerves.

Thus an effective, reliable stimulation system for acupuncture points is not well known.

### SUMMARY OF THE INVENTION

The present invention discloses a system and method for stimulation therapy and therapeutic effect utilizing a stimulation of acupuncture points. The stimulation according to the present invention comprises an electrical stimulation in combination with a laser beam stimulation. The stimulation is preferably in pulse form, such as pulsed electrical current or pulsed laser beam, or a combination of pulsed electrical current and pulsed laser beam. The stimulation can further comprise an electrical voltage stimulation, in addition to an electrical current and laser beam.

The present invention stimulation can further comprise a light radiation, preferably to the vicinity of the acupuncture point to be stimulated. The main purpose of the radiated light is to inhibit stimulation of nearby acupuncture points, thus ensure a stimulation of selective acupuncture points. The radiated light can also serve as an illumination, providing a light source to the area to be stimulated.

The present invention stimulation can further comprise a mechanism to activate the stimulations, with accompanied audible sounds. By asynchronizing/delaying the activation of the stimulations with the accompanied audible sounds, the therapist can control the activity of the stimulation while a pain anticipation of the patient due to the sounds can be avoided.

The present invention can further comprise a stimulation point indicator to assist in the selection of acupuncture points. The stimulation point indicator preferably comprises a display showing a sequence of intended therapy-effect determined to be stimulated regions, distinguishable from other regions. The stimulation point indicator can employ any method of acupuncture point finder, such as the method of measuring skin impedance or resistance in which the acupuncture points have lower skin impedance or resistance than other areas.

The present invention stimulation is preferably controlled by a computer system for reliability, repeatability, and ease of control. Plus, the present invention can be practiced by an operator with a short training on the equipment.

### BRIEF DESCRIPTION OF THE DRAWINGS

FIG. 1 shows an embodiment of the present invention stimulation of acupuncture points, employing a combination of electrical current, laser beam and blue light.

FIG. 2 shows an embodiment of the electrical pulsed current.

### DETAILED DESCRIPTION OF THE INVENTION

In an embodiment of the invention, the system according to the present invention for the stimulation of acupuncture points comprises a power source for delivering an electric current to the acupuncture point, and a laser source for emitting a laser beam, also to the same acupuncture point.

In another embodiment of the invention, the system according to the present invention for the stimulation of acupuncture points comprises a power source for delivering an electric current, a laser source for emitting a laser beam, and a combination unit to enable an overlapping delivery of a combination of the electric current and the laser beam to the acupuncture point.

In one configuration, the laser beam is a pulsed laser beam, and the laser source for emitting a laser beam is set up to emit a pulsed laser beam. The pulsed laser beam is preferably a pulsed red laser light having frequency of 12-18 cycles per second and an intensity of 5-10 mW. The laser beam can have different wavelengths, and the laser source for emitting a laser beam is preferably set up to emit laser beams of different wavelengths.

The power source can be electrically isolated from the main circuit, from the surrounding electrical circuits such as the control computer or the main power supply. The power source is configured for delivering an electrical current, and preferably a pulsed current. The current pulses are preferably having a frequency from 0.5 to 2 cycles per second and amplitude from 0.05 to 3 mA. The current is preferably controlled by a computer for repeatability, accuracy and ease of control.

The combination unit preferably comprises an application housing and a handle. The application housing provides the mechanism for the combination of the laser beam and the electrical current, and preferably comprises an application electrode for delivering the current, with an optical beam guide surrounding the application electrode for conducting the laser light. The application electrode is connected to the power source, and the electrical current is delivered to the stimulation point through this electrode. The optical beam guide is connected to the laser source, and the laser beam is delivered to the stimulation point through this optical guide.

The present invention stimulation system can also comprise an additional source of light, arranged to illuminate the area of acupuncture points. The source of light is preferably a visible light, such as a continuous blue light. The source of light can be arranged above the application housing and radiates a continuous light, preferably blue light, to the vicinity of the acupuncture point to be stimulated. The function of the constant, blue light is a simultaneous inhibition of the surrounding acupuncture points. The light can also serve as illumination for viewing or for camera.

The present invention stimulation system can also comprise an additional stimulation of a DC voltage starting from 140-170 V.

In a preferred embodiment, the body of a patient is connected to the negative terminal of the power source generating the electrical current, or the negative terminal of the DC voltage.

The duration of stimulation can be calculated as a function of biometrics and personal data of the patient.

The activation of the stimulations and the stimulations themselves (such as electrical current stimulation or laser beam stimulation) can be asynchronous, and the asynchronization can be made audible to the user and patient. Thus the activation mechanism of the stimulations according to the present invention stimulation can comprise an audible sound indication which is preferable asynchronous to the stimulation of the electrical current and/or the laser beam. The asynchronization preferably comprises a delay of 20-50 mSec between the application of the stimulations (electrical current and/or laser beam) and the audible signal.

The stimulation system according to invention is preferably accomplished by a computer control for repeatability, reliability and ease of control.

The present invention can further comprise a stimulation point indicator subsystem for acupuncture point therapy in which the body regions which can be stimulated can be indicated as points, and can be distinguishable from other non-stimulationable regions. The stimulation point indicator is preferably displaying therapy adapted stimulationable acupuncture points distinguishable from other non-stimulationable points, or displaying the anatomic locations of a therapy adapted sequence of stimulationable acupuncture points distinguishable from other non-stimulationable points. A stimulation point indicator for acupuncture therapy according to the present invention can comprise indication lines identifying the location of therapy adapted acupuncture points in reference to anatomical landmarks.

The stimulation point indicator can comprise a display with computer-aided control for the display of acupuncture points. The identification of acupuncture points can be accomplished by measuring the skin impedance or resistance. It is known that acupuncture points coincide with points of low body impedance, and thus an impedance measuring system can locate points of low skin resistance or impedance for display or made audible. The stimulation point indicator according to the present invention can greatly simplify the task of the operator servicing the patient by showing on the display the areas that can be stimulated versus the areas that cannot be stimulated. The display of the stimulation point indicator can also display additionally biometrics data of the patient.

The present invention further discloses a stimulation of acupuncture points utilizing a combination of a current and a laser beam and connected with a stimulation point indicator, in particular with the stimulation point indicator system as disclosed above. The acupuncture therapy by a combination of a current/laser beam stimulation in connection with a stimulation point indicator is preferably controlled by a computer. The computer program according to the invention includes program codes to accomplish all the disclosed invention, if the computer program is accomplished on a computer or an appropriate arithmetic and logic unit. The present invention also covers these program codes, which are stored on a computer-readable data medium.

The present invention discloses a system as well as a method for utilizing a combination of a current/laser beam stimulation. The present invention stimulation of acupuncture points by a combination of electrical current stimulation and laser beam stimulation can exhibit therapeutic effect, and can be effective in treatment therapy such as stimulation therapy or acupuncture point therapy. Thus the present invention also discloses a method for stimulation therapy wherein the stimulation is a combination of an electrical current stimulation and laser beam stimulation. The stimulation therapy is preferably applied to an acupuncture point.

A preferred method according to the present invention for the stimulation of an acupuncture point comprises the stimulation of an acupuncture point by a combination of a current and a laser beam.

The therapeutic method according to the present invention also preferably employs a pulsed current, a pulsed laser beam, or a combination of synchronized pulsed current and pulsed laser beam.

In a preferred embodiment, the therapeutic method further comprises a step of applying an additional light illumination, such as a blue light, to the vicinity of the acupuncture point. The additional radiated light can inhibit the nearby acupuncture points to prevent interference. The therapeutic method can further comprise a step of stimulating the acupuncture point with an additional DC voltage from between 140 to 170 V. The therapeutic method can further comprise a step of calculating the duration of the stimulation based on the biometrics and personal data of the patient, and stopping the stimulation when this duration is reached. The therapeutic method can further comprise a step of asynchronizing the activation of the stimulations to the stimulations and making audible the asynchronization to a user or patient. The activation cam be an indication of the stimulation, and the asynchnization can be a time delay, and therefore the step comprises the indication of the stimulation to the stimulation, and then making audible the stimulation to an user or a patient with a delay.

The present invention also comprises the method to display the stimulation regions as points on a display screen, and these stimulation points are distinguishable from other non-stimulationable points. The display is preferably controlled by a computer-aided system for acupuncture therapy, and can further display additional biometrics and personal data of the patient.

The invention, at least with the preferential embodiments, consists of many advantages as listed below.

The stimulations by acupuncture points according to the present invention can be accomplished by persons who do not have training in acupuncture.

The invention, in principle, can be used with all disease or addiction problems, for acupuncture therapy can be an effective treatment on many different illnesses.

The invention allows ease of production and further improvements.

The described pulse waveforms and frequencies are the optimal stimulation form for acupuncture points according to the present invention.

The acoustic support of the asynchronization between the activation of the stimulations and the stimulations can let the user and patient know when the stimulation starts and when it ends. Further, by this asynchronization, a patient's fear of anticipation is prevented and the pain feeling is reduced.

The present invention uses a combination of well-defined electrical currents together with a laser light to accomplish better stimulation sequence of acupuncture points as compared with past procedures. The guidance of the sequences as well as the control of the pulse formation and acupuncture point excitation preferably take place via a computer program, so that a stimulation by acupuncture points is controllable and reproducible as well as optimizable.

The preferred embodiment of the present invention acupuncture therapy is controllable by screen guidance to an operator. This computer-controlled guidance also leads to the reproducibility of the treatment.

Thus an optimal electrical/laser stimulation is disclosed, which leads to provable stimulation results. Further, laser light of different colors can be used. By the combination of the electrical stimulation with pulsed laser light of different colors, an improvement of the stimulation results can further be accomplished.

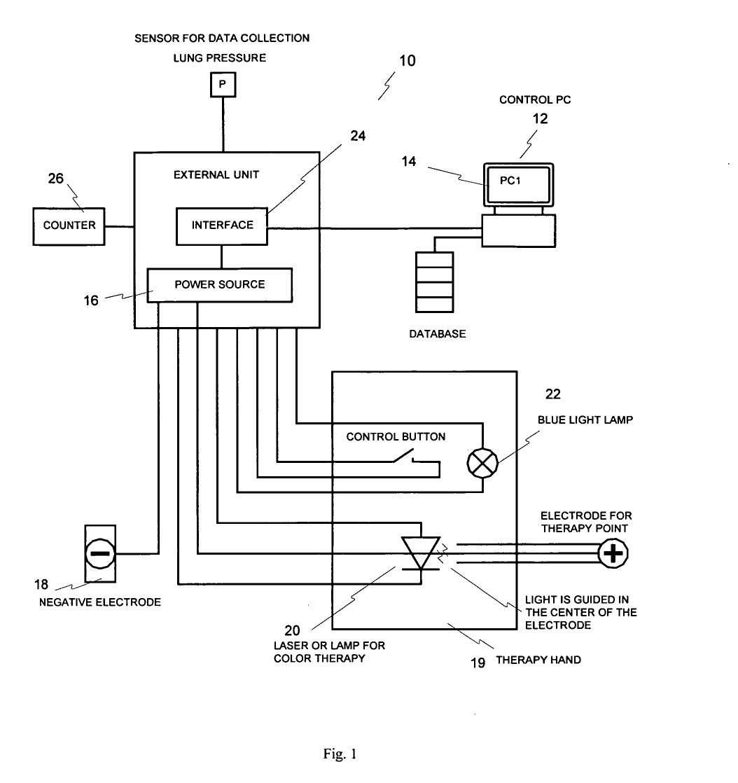

FIG. 1 represents a system 10 according to an embodiment of the present invention. It includes a computer (P/C) 12, on whose monitor 14 the points which can be stimulated are shown, and at which points the optimal stimulation can be performed. The organs illustrated on the monitor 14 are the regions to be treated or the regions where the therapeutic points are lovated (e.g. a knee with degenerative process in a joint, the ears, and the area of the nose wings). In addition lung function data, such as lung pressure collected from sensor P, are shown on the monitor 14. The system 10 can include a conventional impedance measuring system for locating points of low skin resistance or impedance for display on the monitor 14 or audible perception.

The control unit (computer 12 with control program and monitor 14) as shown in FIG. 1 is connected with an external unit through an interface 24. The external unit comprises a power source 16 for delivering the electrical current, and a power source with peripheral components for the laser source. The external unit is controlled by the computer 12 also through the interface 24. The power source 16 is attached to the patient by a hand electrode 18 with negative polarity. The positive terminal is implemented in the form of a combination unit 19 (therapy hand) for the user. The combination unit 19 contains a red light laser 20 (or lamp for color therapy) as well as a blue source of light 22 (a blue light lamp), which also serves as illumination for viewing and photo point search.

Description of the Combination Unit

The combination unit 19 comprises an application housing with a handle. The handle is designed for manual handling of the application housing by an operator. The application housing comprises an electrical conducting material for the electrode carrying the electrical current, and is surrounded by an optical conductor guide from the red laser device. The red laser light is positioned in the center of the electrode contact surface and beams direct at the patient, together with the electrical current from the electrode. The laser light is pulsed and can be switched on or off separately. Above the application housing is a source of light 22 for continuous blue light, which can also be switched on and off separately. The function of the pulsating, red laser light is an activation of the underlying acupuncture points in the skin of the patient. The function of the constant, blue light is a simultaneous inhibition of the surrounding acupuncture points. The electrode for therapy point possesses a spherical contact surface of approx. 5 mm in diameter. This ensures a safe transmission of the current pulse and also serves as a cover of the acupuncture point from the irradiation of the blue light, which inhibits other nearby acupuncture points from being stimulated. The stimulation of the respective acupuncture point is started by the user by a control button at the combination unit, which is connected with the computer 12. The computer automatically controls the time of stimulation per simulation point according to the biometrics and personal data of the patient. In addition, the control button provides a means for any interruption and restart of the stimulation by the user at any time.

Description of the Power Source

The stimulation system comprises a power source 16, which is connected by an interface 24 to the computer 12. The power source 16 is electrically isolated from the surrounding circuits and from the computer 12. It can have voltage and current limiters, generating a maximum voltage and a maximum current. The computer 12 controls the magnitude of the current, the duration and the waveform of the current pulse as well as the duration of the synchronous, pulsed laser beam. This control is passed on to the combination unit 19. At the computer interface, sensors (e.g. lung pressure P, pressure sensors for breath current measurements, CO measurement, and/or other measuring instrument) are also attached for the collection by biometrics data.

Description of the Stimulation Point Indicator

The user enters first the numeric data of the patient relevant to the stimulation. Additionally biometrics measured values can be displayed over the interface 24 (e.g. max. lung pressure P). The software computed the optimal duration of the stimulation from the biometrics measured values and the entered patient data and then displays this information on the monitor 14. The stimulation treating stops the current intensity and/or the laser beam when reaching the optimal duration as calculated and shown on the monitor 14. The monitor screen surface shows the relevant regions (e.g. ears, nose, knee joints etc.) with the points which can be stimulated. The points that can be stimulated in each case are indicated distinctive to the other non-stimulationable points on the monitor 14. The stimulation of the succession of the points is consistently controlled by the software. A mechanical counter 26 can be used to control the duration of the stimulation or the number of stimulation points through the interface 24. Thus the number of accomplished stimulations can be ascertained.

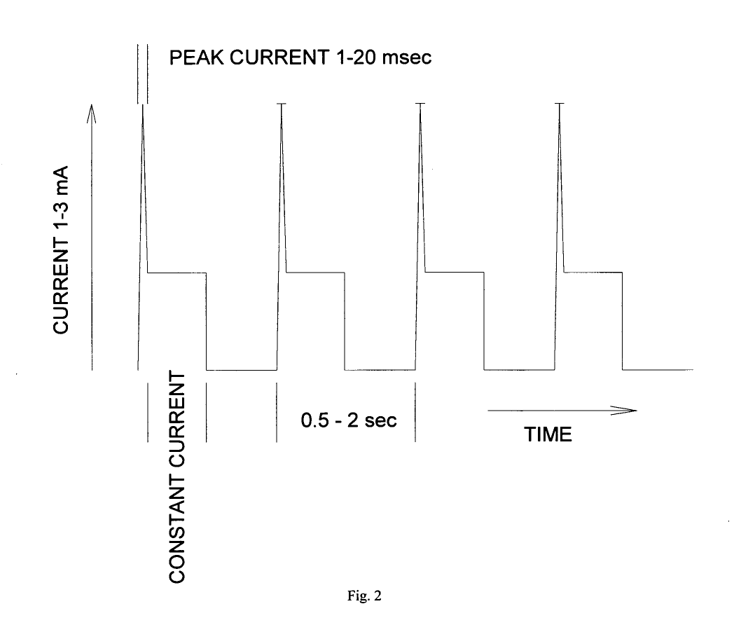

FIG. 2 shows a preferred embodiment of the electrical current waveform of one impulse. The period of the electrical current is about 0.5 to 2 sec, with a peak current of less than 20 msec, preferably between 1 to 20 msec. The peak current is less than 3 mA in magnitude, preferably between 0.05 and 3 mA. A constant current is then applied after the initial peak current. The constant current can last for the rest of the period, or can last only partially (about half of the period as shown in FIG. 2), with another constant current (preferably zero current) for the rest of the period

What is claimed is:

1. A system for stimulation of an acupuncture point, comprising
a power source for delivering an electrical current;
a laser source for emitting a laser beam; and
a combination unit to enable an overlapping delivery of a combination of the electric current and the laser beam to the acupuncture point.

2. A system of claim 1, wherein the laser source is configured to emit a pulsed laser beam.

3. A system of claim 1, wherein the laser source is configured to emit laser beams of different wavelengths.

4. A system of claim 1, wherein the power source is configured to deliver a pulsed current.

5. A system of claim 1, further comprising a source of light.

6. A system of claim 5, wherein the source of light inhibits other surrounding acupuncture points from being stimulated.

7. A system of claim 1, further comprising a DC voltage source from between 140-170 V for delivering an electrical voltage to the acupuncture point.

8. A system of claim 1, further comprising an activation mechanism to activate the stimulations, wherein the activation mechanism comprises an audible indication of the stimulations which is asynchronous to the stimulation of the electrical current and the laser beam.

9. A system of claim 8, wherein the asynchronization comprises a delay of 20-50 mSec between the application of the stimulation and the audible signal.

10. A system of claim 1, further comprising a stimulation point indicator, the stimulation point indicator displaying stimulationable acupuncture points distinguishable from other non-stimulationable points.

11. A therapy system for stimulation of an acupuncture point, comprising a combination of
a power source for delivering an electrical current; and
a laser source for emitting a laser beam,
wherein the electrical current and the laser beam are delivered overlappingly to the acupuncture point.

12. A system of claim 11 further comprising a source of radiated light for radiating a vicinity of the acupuncture point.

13. A system of claim 11 wherein radiated light inhibits the nearby acupuncture points from being stimulated.

14. A system of claim 11, further comprising a stimulation point indicator, the stimulation point indicator displaying therapy adapted stimulationable acupuncture points distinguishable from other non-stimulationable points.

15. A system of claim 14, wherein the distinction of stimulationable regions and non-stimulationable regions is based on skin impedance or skin resistance.

16. A system for selective stimulation of an acupuncture point, comprising
a power source for delivering an electrical current to the acupuncture point;
a laser source for emitting a laser beam to the acupuncture point;
wherein the acupuncture point is stimulated by the combination of the electrical current and the laser beam, and
a light source for radiating a radiated light to a vicinity of the acupuncture point;
wherein the radiated light inhibits the stimulation of nearby acupuncture points.

17. A system of claim 16, further comprising a stimulation point indicator, the stimulation point indicator displaying the anatomic locations of a therapy adapted sequence of stimulationable acupuncture points distinguishable from other non-stimulationable points.

18. A stimulation point indicator for acupuncture therapy, the indicator comprising a display wherein regions which can be stimulated in acupuncture therapy are indicated as points on the display, and wherein such indicated points are differentiable from other regions which are not to be stimulated.

19. A stimulation point indicator for acupuncture therapy, the indicator comprising indication lines identifying the location of therapy adapted acupuncture points in reference to anatomical landmarks.

20. An indicator of claim 19, wherein the display further showing of additional biometrics data.

21. A computer program to control the overlapping delivery of an electrical current and a laser beam to an acupuncture point in a therapy using stimulation of an acupuncture point.
